(19) United States
(12) Patent Application Publication
(10) Pub. No.: US 2017/0215772 A1
(43) Pub. Date: Aug. 3, 2017

(54) METHOD AND DEVICE FOR DETERMINING THE TIME CURVE OF THE DEPTH OF BREATH

(71) Applicant: AIT AUSTRIAN INSTITUTE OF TECHNOLOGY GMBH, WIEN (AT)

(72) Inventors: HEINRICH GARN, WIEN (AT); BERNHARD KOHN, WIEN (AT); CHRISTOPH WIESMEYR, WIEN (AT)

(21) Appl. No.: 15/509,936

(22) PCT Filed: Sep. 9, 2015
(86) PCT No.: PCT/EP2015/070608

§ 371 Date: Mar. 9, 2017

(30) Foreign Application Priority Data
Sep. 10, 2014 (DE) 10 2014 218 140.2

(51) Int. Cl. A61B 5/113; A61B 5/08; A61B 5/11

(52) U.S. Cl. CPC A61B 5/1135; A61B 5/113; A61B 5/1128; A61B 5/0806; A61B 5/08

(57) ABSTRACT

A method and a device determine a time curve of the depth of breath of a sleeping person. Height profiles of the person at individual recording time points are continuously determined. Height profiles from adjacent recording time points are combined to give segments. The region which indicates the abdomen or chest region of the person depending on a corresponding reference point or reference region is selected as an observation region. For each height profile within the segment, the corresponding average value of the distances of the points of the height profile, which points lie within the observation region, from a reference point is determined. For the segment, a signal is determined and for each recording time point, the average value determined for this height profile is associated with the signal. On the basis of the determined signal, values which characterize the time curve of the depth of breath are determined.

The invention relates to a method for determining time curve of the depth of respiration of a person, in particular a sleeping person. Furthermore, the invention relates to an apparatus for determining the time curve of the depth of respiration. Finally, the invention relates to a data medium comprising a program for determining the time curve of the depth of respiration.

Height profiles of the person are created at individual recording times on an ongoing basis.

Height profiles from adjacent recording times are combined to form segments. The region which specifies the abdominal or chest region of the person depending on the respective reference point or reference region is selected as observation region.

The mean value of the distances of the points of the height profile situated within the observation region from a reference point or reference object is ascertained, separately in each case, for each height profile within the segment. A signal is ascertained for the segment, the mean value ascertained for the height profile at the respective recording time of the height profile being assigned to said signal.

One or more values characterizing the time curve of the depth of respiration are ascertained on the basis of the ascertained signal, in particular on the basis of the signal amplitude thereof.

The prior art has disclosed a multiplicity of methods with which the respiratory activity of a person is recorded, in particular during sleep. In these known methods, the person to be examined is in each case connected directly to various different sensors, which may disturb the person during sleep. The cables and lines sometimes used for immediate evaluation of the sensor signals may also disturb the sleep of the relevant person.

Therefore, the problem addressed by the invention is that of providing a method and an apparatus which do not disturb the person to be examined much and, in particular, which determine the respiration in a contactless manner.

The invention solves this problem in respect of the method specified at the outset by way of the features of patent claim 1. The invention solves this problem in respect of the apparatus specified at the outset by way of the features of patent claim 18.

In a method for determining the time curve of the depth of respiration of a person, in particular a sleeping person, the invention provides for a detector unit directed to the person to be used on an ongoing basis for in each case creating a height profile of the person at successive recording times,

a) wherein a number of at least two points are set in space in the height profile, said points lying on the surface of the person or on the surface of an object situated on, or next to, the person,
b) wherein the respective height profile for each of the recording times is stored and kept available in a data structure,
c) wherein a number of height profiles recorded at successive recording times, in particular within a time range of 3 to 20 seconds, are combined to form a segment,
d) wherein a region which specifies the abdominal or chest region of the person depending on the respective reference point or reference region is selected as observation region,
e) wherein the mean value of the distances of the points of the height profile situated within the observation region from a reference point or reference object is ascertained, separately in each case, for each height profile within the segment and a signal is ascertained for the segment, the mean value ascertained for the height profile at the respective recording time of the height profile being assigned to said signal, and
f) wherein one or more values characterizing the time curve of the depth of respiration is or are ascertained on the basis of the ascertained signal, in particular on the basis of the signal amplitude thereof.

These advantageous measures facilitate contactless detection of the respiration of a person, which does not disturb the sleep of the person to be examined. In particular, it is not necessary to fasten sensors to the body of the person to be examined.

An advantageous and numerically simple embodiment of the invention provides for determining the time curve of the depth of respiration of a person, in particular a sleeping person, wherein a detector unit directed to the person is used on an ongoing basis for in each case creating a height profile of the person at successive recording times,

a) wherein the height profile has a number of at least two distance values for in each case setting one point in space, wherein the individual distance values in each case specify the distance of the point of intersection of a beam and the surface of the person or the surface of an object situated on the, or next to the, person from a reference point or a reference plane, said beam being set in advance relative to the detector unit and, in particular, emanating from the detector unit,
b) wherein a data structure is created in each case for each of the recording times, said data structure containing the respective height profile, wherein all data structures created thus have the same size in each case and each have memory positions for the individual distance values of the height profile,
c) wherein a number of height profiles recorded at successive recording times, in particular within a time range of 3 to 20 seconds, are combined to form a segment,
d) wherein a number of memory positions in the data structure, in which distance values specifying the distance of the abdominal or chest region of the person depending on the respective reference point or reference region are stored, are selected as observation region,
e) wherein the mean value of the distance values situated within the observation region is ascertained, separately in each case, for each height profile within a segment and a signal is ascertained for the segment, the mean value ascertained for the height profile at the respective recording time of the height profile being assigned to said signal, and
f) wherein one or more values characterizing the time curve of the depth of respiration is or are ascertained on the basis of the ascertained signal, in particular on the basis of the signal amplitude thereof.

An alternative advantageous and numerically precise embodiment of the invention, which facilitates the use of height profiles in the form of general point clouds, provides for the height profile to describe a point cloud with a number of at least two points in space, said points lying on the surface of the person or on the surface of an object situated on, or next to, the person.

An advantageous extraction of the depth of respiration from the signal is achieved by virtue of at least one maximum and at least one minimum being extracted from the signal for the purposes of characterizing the depth of respiration in a segment and at least one difference between the maximum and the minimum being used as value characterizing the depth of respiration for the time range assigned to the segment.

An alternative advantageous extraction of the depth of respiration from the signal is achieved by virtue of the signal being subjected to a spectral transformation, in particular a Fourier transform or a cosine transform or wavelet transform, and the spectral component with the highest signal energy being searched for within a predetermined frequency band, in particular from 0.1 Hz to 1 Hz, and the signal energy of this spectral component being used to characterize the depth of respiration in this segment.

For the purposes of the low-noise extraction of respiratory movements, from which, ultimately, a particularly good extraction of the depth of respiration is possible, provision may be made for the signal assigned to a segment to be subjected to noise filtering after the creation thereof, in particular prior to determining the value characterizing the depth of respiration, wherein, in particular,

a) signal components with a frequency of more than 0.5 Hz are suppressed and/or
b) direct components are suppressed.

The curve of the depth of respiration over time may advantageously be ascertained by virtue of the depth of respiration being ascertained separately in each case for a number of overlapping or non-overlapping segments.

An advantageous adaptive adaptation of the observation region, which may regularly be undertaken with little outlay, provides for the observation region for each segment to be ascertained separately, in particular on the basis of the observation region ascertained for the respective preceding segment.

Particularly meaningful height profiles may be created by virtue of—the height profile being characterized by a two-dimensional matrix data structure comprising a number of rows and columns,
a number of positions arranged in a grid-shaped manner in lines and columns being predetermined, with the distance values of the height profile being determined at said positions in each case,
the matrix data structure having a grid with the same size and structure, and
the matrix data structure being created by virtue of the distance values recorded at the respective positions being stored and being kept available at the memory positions, corresponding to the positions in the grid, in the matrix data structure.

In order to facilitate faster processing of the data, a reduction in the required storage space and a reduction in the occurring noise, provision may be made for the matrix data structure, after the creation thereof, to be replaced in its dimensions by a reduced matrix data structure, wherein a mean distance value is ascertained in each case for rectangular and non-overlapping image regions, which in particular cover the entire matrix data structure, having the same size in the matrix data structure in each case and this mean distance value to be assigned to the image point, corresponding in terms of the position thereof, in the reduced matrix data structure.

An advantageous method for determining the observation region provides for the observation region to be placed by virtue of

a) a number of possible observation regions being predetermined in advance in the height profile, in particular in the matrix data structure or in the point cloud,
b) the respective depth of respiration being ascertained for the respective segment on the basis of each one of the possible observation regions, and
c) the predetermined possible observation region with the largest ascertained depth of respiration being used as observation region.

A second advantageous method for determining the observation region, which particularly takes account of anatomical conditions, provides for the observation region to be placed by virtue of

a) regions being searched for in the height profile, in particular in the matrix data structure or in the point cloud, by means of object recognition, or in that regions are selected in advance, said regions corresponding to a human head and torso, and
b) the region of the height profile imaging the torso, in particular the region of the matrix data structure or of the point cloud, or a portion of this region being selected as observation region, wherein, in particular,
the portion of this region close to the head is selected as observation region for the purposes of detecting the curve or the depth of respiration of the costal respiration and/or
the portion of this region away from the head is selected as observation region for the purposes of detecting the curve or the depth of respiration of the diaphragmatic respiration.

A fast adaptation of the observation region may be achieved by virtue of, for each segment to be examined or for individual height profiles of a segment, the observation region being adaptively ascertained anew by means of object recognition applied to the height profiles present in the segment, proceeding from the position of the selected observation region in the respectively preceding segment.

A third advantageous method for determining the observation region, which can be carried out in a numerically stable and efficient manner, provides for the observation region to be placed by virtue of

a) the variance over the respective segment being determined separately for all points of the height profile, in particular for all entries of the matrix data structure or the point cloud, and
b) one region or a plurality of regions with respectively contiguous points of the height profile, the respective variance of which lies above a lower threshold or within a predetermined interval, being selected as observation region, in particular
by virtue of contiguous regions of points being selected as observation region, the size of which exceeds a predetermined number of points,
wherein, in particular, a region is considered to be contiguous if each point of the region is reachable via respectively adjacent pixels proceeding from another point within this region,
wherein, preferably, points of the height profile are considered to be adjacent
   if they are set by adjacent entries of the matrix data structure or
   if they, or the projection thereof onto a horizontal plane in space, have a distance from one another which is less than a threshold.

In order to be able to detect situations in which the person to be examined moves and in order to be able to discard the erroneous measurement values recorded in these situations, provision may be made for the observation region to be ascertained separately in two or more successive segments and for a height profile to be discarded and an error message to be output if the observation region, in particular the size and/or the centroid thereof, is displaced by a predetermined threshold in relation to the respective preceding segment.

For the purposes of an improved detection of the interruption of respiration, acoustic monitoring may additionally be provided. Here, provision is made for

a) the sound emitted by the person to be ascertained simultaneously and in parallel with the distance of the person from the detector unit being recorded, said sound being kept available in the form of an audio signal with a number of audio sampling values,
b) the audio signal to be subdivided into a number of audio segments, in particular with a length from 100 ms to 1 second, and for an examination to be carried out for each of the audio segments as to whether it contains human respiratory noises or other noises, and for a classification result to be respectively kept available for each audio segment,
c) audio segments and segments recorded at the same time to be assigned to one another, and
d) audio segments or segments with a low depth of respiration or a lack of respiratory noises to be searched for and for the segments or audio segments assigned to such an audio segment or segment to be likewise examined for the presence of a low depth of respiration or lack of respiratory noises, and
e) the lack of respiration of the person to be determined should a low depth of respiration and a lack of respiratory noises be detected in each case in segments and audio segments that are assigned to one another.

A particularly preferred embodiment of the invention, which facilitates a detection of the start and the end of the interruption of respiration, provides for

a) a classification signal to be created, said signal in each case containing at least one classification result for each audio segment, said classification result specifying the type and strength of the respective noise in the time range of the respective audio segment,
b) for in each case a number of successive audio segments to be combined to form further audio segments, in particular with a duration of 5 to 10 seconds, which preferably comprise the same time ranges as the segments,
c) averaging to be undertaken over the classification results contained within a further audio segment and the respective mean value to be assigned to the further audio segment,
d) a further classification signal to be created by interpolating the mean values of the further audio segments,
e) times at which strong changes occur in both signals to be searched for in the further classification signal and in the depth of respiration signal,
f) the times identified thus to be classified as ever more relevant with increasing strength of the change in the respective signal at the respective time,
g) a relevance value in this respect to be assigned to these times in each case, and
h) the times for which the magnitude of the relevance value lies above the magnitude of a threshold to be detected as start points or end points of apnea, wherein, in particular,
the threshold is formed by virtue of a mean value of the reference measure being formed over a time range, in particular before and/or after the comparison time with the reference measure, and the threshold being set in the range between 120% and 200% of this mean value.

A method for carrying out a method according to the invention may advantageously be stored on a data medium.

The invention further relates to an apparatus for determining the time curve of the depth of respiration of a person, in particular a sleeping person, comprising
a detector unit alignable onto a space to sleep, in particular onto a bed, said detector unit, on an ongoing basis, in each case creating a height profile of the person for successive recording times, wherein a number of at least two points are set in space in the height profile, said points lying on the surface of the person or on the surface of an object situated on, or next to, the person,
   and
a processing unit, which
   a) stores and keeps available the respective height profile in a data structure for each of the recording times,
   b) combines a number of height profiles recorded at successive recording times, in particular within a time range of 3 to 20 seconds, to form a segment,
   c) selects as observation region a region which specifies the abdominal or chest region of the person depending on the respective reference point or reference region,
   d) ascertains the mean value of the distances of the points of the height profile situated within the observation region from a reference point or reference object, separately in each case, for each height profile within the segment, and a signal is ascertained for the segment, the mean value ascertained for the height profile at the respective recording time of the height profile being assigned to said signal, and
   e) ascertains a value characterizing the depth of respiration, in particular subsequently, on the basis of this signal or the signal amplitude thereof.

These advantageous measures facilitate contactless detection of the respiration of a person, which does not disturb the sleep of the person to be examined. In particular, it is not necessary to fasten sensors to the body of the person to be examined.

An advantageous and numerically simple embodiment of the invention provides for an apparatus for determining the time curve of the depth of respiration of a person, in particular a sleeping person, comprising
a detector unit alignable onto a space to sleep, in particular onto a bed, said detector unit, on an ongoing basis, in each case creating a height profile of the person for successive recording times, wherein the height profile has a number of at least two distance values for in each case setting one point in space, wherein the individual distance values in each case specify the distance of the point of intersection of a beam and the surface of the person or the surface of an object situated on the, or next to the, person from a reference point or a reference plane, said beam being set in advance relative to the detector unit and, in particular, emanating from the detector unit,
   and
a processing unit, which
   a) creates a data structure in each case for each of the recording times, said data structure containing the respective height profile, wherein all data structures created thus have the same size in each case and each have memory positions for the individual distance values of the height profile,
   b) combines a number of height profiles recorded at successive recording times, in particular within a time range of 3 to 20 seconds, to form a segment,
   c) selects a number of memory positions in the data structure, in which distance values specifying the distance of the abdominal or chest region of the person depending on the respective reference point or reference region are stored, as observation region,
   d) ascertains the mean value of the distance values situated within the observation region, separately in each case, for each height profile within a segment and ascertains a signal for the segment, the mean value ascertained for the height profile at the respective recording time of the height profile being assigned to said signal,
   e) ascertains a value characterizing the depth of respiration, in particular subsequently, on the basis of this signal or the signal amplitude thereof.

An alternative advantageous and numerically precise embodiment of the invention, which facilitates the use of height profiles in the form of general point clouds, provides for the detector unit to create the height profiles in the form of point clouds with a number of at least two points in space, wherein the points lie on the surface of the person or on the surface of an object situated on, or next to, the person.

In an apparatus for determining the time curve of the depth of respiration of a person, in particular a sleeping person, the invention provides for the processing unit to extract at least one maximum and at least one minimum from the signal for the purposes of characterizing the depth of respiration in a segment and keep available and, where required, use the at least one difference between the maximum and the minimum as value characterizing the depth of respiration for the time range assigned to the segment.

A numerically simple filtering of the signal, which largely suppresses interfering noise, provides for the processing unit to subject the signal to a spectral transformation, in particular a Fourier transform, a cosine transform or wavelet transform, and search for the spectral component with the highest signal energy within a predetermined frequency band, in particular from 0.1 Hz to 1 Hz, and use the signal energy of this spectral component to characterize the depth of respiration in this segment.

For the purposes of the low-noise extraction of respiratory movements, from which, ultimately, a particularly good extraction of the depth of respiration is possible, provision may be made for the processing unit to subject the signal assigned to a segment to noise filtering after the creation thereof, in particular prior to determining the value characterizing the depth of respiration, wherein, in particular, said processing unit

a) suppresses signal components with a frequency of more than 0.5 Hz and/or
b) suppresses direct components.

The curve of the depth of respiration over time may advantageously be ascertained by virtue of the processing unit ascertaining the depth of respiration separately in each case for a number of overlapping or non-overlapping segments.

An advantageous adaptive adaptation of the observation region, which may regularly be undertaken with little outlay, provides for the processing unit to ascertain the observation region for each segment separately, in particular on the basis of the observation region ascertained for the respective preceding segment.

Particularly meaningful height profiles which are numerically easy to manage may be created by virtue of the processing unit
characterizing the height profile by a two-dimensional matrix data structure comprising a number of rows and columns,
predeterming a number of positions arranged in a grid-shaped manner in lines and columns, with the distance values of the height profile determined at said positions in each case,
the matrix data structure having a grid with the same size and structure, and
creating the matrix data structure by virtue of it storing and keeping available the distance values recorded at the respective positions at the memory positions, corresponding to the positions in the grid, in the matrix data structure.

In order to facilitate faster processing of the data, a reduction in the required storage space and a reduction in the occurring noise, provision may be made for the processing unit to replace the matrix data structure, after the creation thereof, in its dimensions by a reduced matrix data structure,

wherein it ascertains a mean distance value in each case for rectangular and non-overlapping image regions, which in particular cover the entire matrix data structure, having the same size in the matrix data structure in each case and assigns this mean distance value to the image point, corresponding in terms of the position thereof, in the reduced matrix data structure.

An advantageous method for determining the observation region provides for the processing unit to set the observation region by virtue of it

a) predetermining a number of possible observation regions in advance in the in the height profile, in particular in the matrix data structure or in the point cloud,
b) ascertaining the respective depth of respiration for the respective segment on the basis of each one of the possible observation regions, and
c) selecting and using the predetermined possible observation region with the largest ascertained depth of respiration as observation region.

A second advantageous method for determining the observation region, which particularly takes account of anatomical conditions, provides for the processing unit to set the observation region by virtue of it

a) searching for regions in the height profile, in particular in the matrix data structure or in the point cloud, by means of object recognition, or providing means for selecting regions, said regions corresponding to a human head and torso, and
b) selecting the region of the the height profile imaging the torso, in particular the region of the matrix data structure or of the point cloud, or a portion of this region as observation region, wherein, in particular, it
selects the portion of this region close to the head as observation region for the purposes of detecting the curve or the depth of respiration of the costal respiration and/or
selects the portion of this region away from the head as observation region for the purposes of detecting the curve or the depth of respiration of the diaphragmatic respiration.

A fast adaptation of the observation region may be achieved by virtue of the processing unit adaptively ascertaining, for each segment to be examined or for individual height profiles of a segment, the observation region anew by means of object recognition applied to the height profiles present in the segment, proceeding from the position of the selected observation region in the respectively preceding segment.

A third advantageous method for determining the observation region, which may be carried out in a numerically stable and efficient manner, provides for the processing unit to set the observation region by virtue of it

a) separately determining the variance of the values over the respective segment for all points of the height profile, in particular for all entries of the matrix data structure, and
b) selecting one region or a plurality of regions with respectively contiguous points of the height profile, the respective variance of which lies above a lower threshold or within a predetermined interval, as observation region, in particular
by virtue of it selecting contiguous regions as observation region, the size of which exceeds a predetermined number of points,
wherein, in particular, a region counts as contiguous if each point of the region is reachable via respectively adjacent pixels proceeding from another point within this region,
wherein, preferably, points of the height profile count as adjacent
   if they are set by adjacent entries of the matrix data structure or
   if they, or the projection thereof onto a horizontal plane in space, have a distance from one another which is less than a threshold.

In order to be able to detect situations in which the person to be examined moves and in order to be able to discard the erroneous measurement values recorded in these situations, provision may be made for the processing unit to ascertain the observation region separately in two or more successive segments and discard a height profile and, when necessary, output an error message if the observation region, in particular the size and/or the centroid thereof, is displaced by a predetermined threshold in relation to the respective preceding segment.

For the purposes of an improved detection of the interruption of respiration, acoustic monitoring may additionally be provided. Here, provision is made for a microphone to be disposed upstream of the processing unit, said microphone keeping available in the form of an audio signal at the output thereof the sound emitted by the person simultaneously and in parallel with the recording of the distance of the person, and this audio signal is supplied to the processing unit, and for the processing unit to

a) subdivide the audio signal into a number of audio segments, in particular with a length from 100 ms to 1 second, and examine for each of the audio segments as to whether human respiratory noises or other noises can be heard therein, and respectively keep available a classification result for each audio segment,
c) assign audio segments and segments recorded at the same time to one another, and
d) search for audio segments or segments with a low depth of respiration or a lack of respiratory noises and likewise examines the segments or audio segments assigned to such an audio segment or segment for the presence of a low depth of respiration or lack of respiratory noises, and
e) determine the lack of respiration of the person should a low depth of respiration and a lack of respiratory noises be detected in each case in segments and audio segments that are assigned to one another.

A particularly preferred embodiment of the invention, which facilitates a detection of the start and the end of the interruption of respiration, provides for the processing unit to

a) create a classification signal, said signal in each case containing at least one classification result for each audio segment, said classification result specifying the type and strength of the respective noise in the time range of the respective audio segment,
b) in each case combine a number of successive audio segments to form further audio segments, in particular with a duration of 5 to 10 seconds, which preferably comprise the same time ranges as the segments,
c) undertake averaging over the classification results contained within a further audio segment and assign the respective mean value to the further audio segment,
d) create a further classification signal by interpolating the mean values of the further audio segments,
e) search for times at which strong changes occur in both signals in the further classification signal and in the depth of respiration signal, and
classify the times identified thus as ever more relevant with increasing strength of the change in the respective signal at the respective time, wherein it assigns a relevance value in this respect to these times in each case, and
f) detects the times for which the magnitude of the relevance value lies above the magnitude of a threshold as start points or end points of apnea, wherein, in particular, it
forms the threshold by virtue of it forming a mean value of the reference measure over a time range, in particular before and/or after the comparison time with the reference measure, and setting the threshold in the range between 120% and 200% of this mean value.

A plurality of exemplary embodiments and variants of the invention are described in more detail on the basis of the following figures in the drawing.

FIG. 1 shows a bed with a sleeping person.

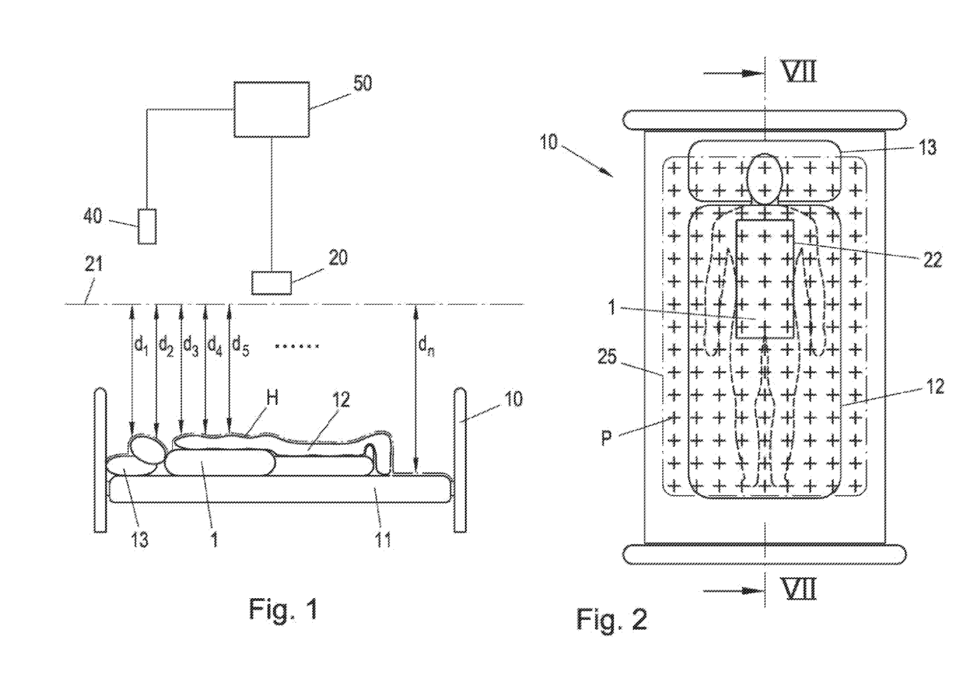

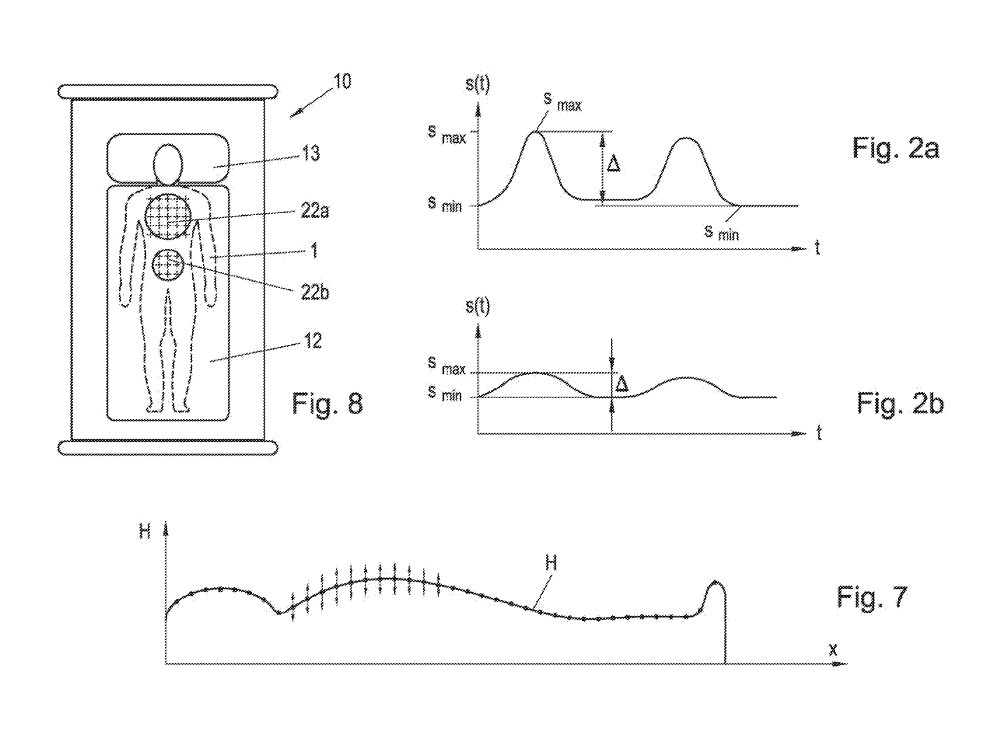

FIG. 2 shows a view of the bed depicted in FIG. 1 from above. FIG. 2a shows a signal curve of the signal in the case of a person with normal respiration. FIG. 2b shows the signal curve of the signal in the case of a person with a respiratory obstruction.

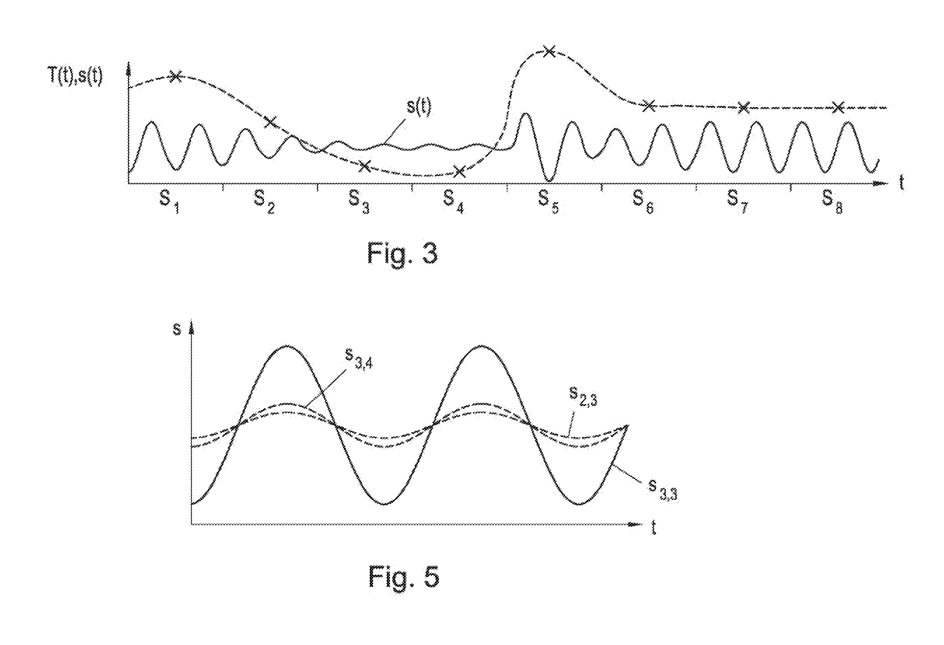

FIG. 3 shows a sequence of signals of segments which are successive in time.

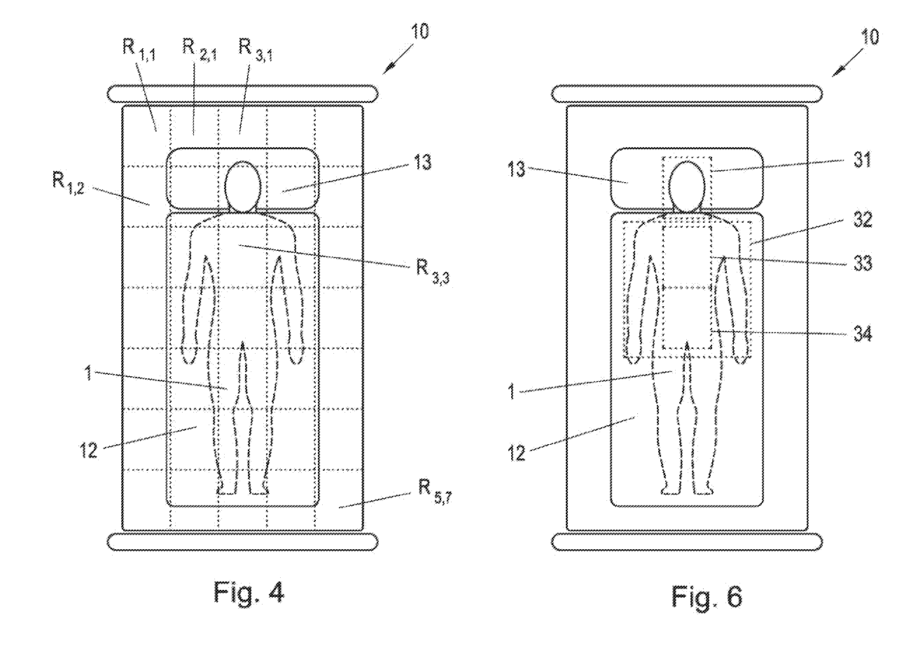

FIG. 4 and FIG. 5 show a first procedure for determining the observation region.

FIG. 6 shows an alternative procedure for determining the observation region with the aid of object recognition algorithms.

FIG. 7 and FIG. 8 show a third procedure for determining the observation region.

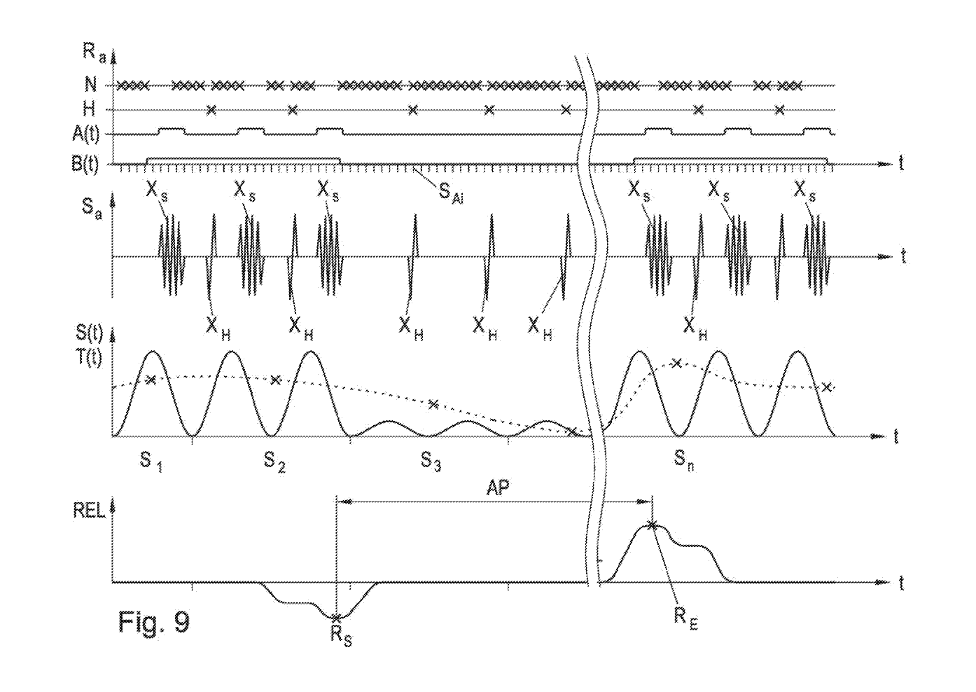

FIG. 9 shows a combination of a method based on the evaluation of height profiles with an acoustic method for identifying interruptions of respiration.

FIG. 1 shows a bed 10 from the side. The bed 10 has a body, a mattress 11, a pillow 13 and a blanket 12. A sleeping person 1, whose head lies on the pillow 13 and whose upper body and legs are covered by the blanket 12, lies in the bed 10.

Arranged above the person there is a detector unit 20, by means of which the distance of the person 1, or of a multiplicity of points on the person, from a predetermined position set relative to the detector unit 20 may be ascertained. Disposed downstream of the detector unit 20 there is a processing unit 50 which carries out the numerical processing steps illustrated below.

In the present case, the detector unit 20 is a unit which in each case specifies the normal distance of a point on the person 1 from a reference plane 21 extending horizontally above the person 1. The detector unit 20 measures the respective distance d₁, . . . , dₙ at n different positions, which are arranged in a grid-shaped manner in the form of lines and columns in the present exemplary embodiment, and creates a height profile H on the basis of these distance measurement values.

In the present exemplary embodiment, a detector unit 20 arranged approximately 2 meters above the person is used for creating a height profile H. Such a detector unit may have different designs.

In a first embodiment variant, the detector unit may be embodied as a time-of-flight camera (TOF camera). It determines the distance from an object with the aid of the “time-of-flight” of an emitted light pulse. In the process, distance measurements with a lateral resolution typically of the order of approximately 320×240 pixels arise. The specific functionality of such a TOF camera is known from the prior art and illustrated in more detail in Hansard, M., Lee, S., Choi, O., and Horaud, R. (2013), Time-of-fight cameras, Springer.

Alternatively, a height profile may also be determined by means of light section methods. Therein, the surface is triangulated with the aid of a light source directed onto the person 1. It is not absolute distance measurements, but only a height profile that is obtained. Absolute measurement values are not used within the scope of the invention in any case; instead, it is only the relative values such as variance or the change in variance over time which are used, and so height profiles may readily be used in the invention as well. The following publication describes a system which supplies height profiles with a frame rate of approximately 40 fps and with a resolution of 640×480 pixels: Oike, Y., Shintaku, H., Takayama, S., Ikeda, M., and Asada, K. (2003). Real-time and high-resolution 3d imaging system using light-section method and smart cmos sensor, In Sensors, 2003, Proceedings of IEEE, volume 1, pages 502-507, IEEE.

A further alternative method for recording a height profile uses a radar measurement. To this end, use is made of radar measuring devices, optionally radar measuring devices which are controllable in terms of the direction thereof, i.e. so-called phased arrays. With the aid of phase shifts in the radar pulse in the antenna array, the pulse may be directed to a certain point on the body of the person 1 and the space may be sampled therewith. As a matter of principle, a high spatial resolution may be obtained using such a radar measuring device. It is also readily possible to obtain 30 height profiles per second. The specific design of such radar measuring devices is described in Mailloux, R. J. (2005), Phased array antenna handbook, Artech House Boston.

FIG. 2 shows a view from above of the bed 10 depicted in FIG. 1, with the distance d₁, . . . , dₙ of the person 1 and/or of the blanket 12 covering the person 1 and/or of the pillow 13 from the reference plane 21 being measured in each case at a number n of positions. The individual positions at which measurements are carried out are arranged in a grid-shaped manner and depicted by crosses in FIG. 2. These positions may be uniquely described by specifying an x-coordinate and a y-coordinate. A height profile H of the person 1 is created in an ongoing manner at successive recording times t₁, . . . , tₚ from the individual ascertained distances d₁, . . . , dₙ. In the present case, the height profile contains the individual ascertained distances d₁, . . . , dₙ and is kept available in the form of a matrix data structure.

However, such representation of the height profile is not mandatory for the invention for a number of reasons.

The height profile need not necessarily specify the distance of the surface of the person or the surface of an object situated on the, or next to the, person from a reference plane 21 at points which are arranged in a lateral grid-shaped manner. Rather, it is also conceivable for the distances to be measured along predetermined beams which emanate from the detector unit and for the vertical distances from the respective point of intersection with the surface to the reference plane then to be calculated with the aid of the measured distance and the respectively used measurement angle. Here, the angles of the various measurement beams may be selected in such a way that a grid-shaped arrangement would emerge upon incidence on a plane lying parallel to the reference plane 21. Even in the case of this very specific selection of measurement beams, lateral measurement points emerge, the x- and y-coordinates of which deviate from the regular grid arrangement in FIG. 2 as soon as the measured surface deviates from a plane. However, since the variations in the height of the measured surface of an object situated on the, or next to the, person are typically small relative to the distance from the detector unit, the lateral coordinates in the aforementioned arrangement of the measurement beams may be assumed to be approximately in the arrangement of FIG. 2 and hence in a matrix data structure.

In general, the individual distance values each specify the distance of the point of intersection of a beam and the surface of the person (1) or the surface of an object situated on the, or next to the, person (1) from a reference point or a reference plane (21), said beam being set in advance relative to the detector unit and, in particular, emanating from the detector unit.

Further, it is not necessary for the individual available distance values to be arranged in a matrix data structure. It is also possible for a data structure with a deviating design to be selected and for distances only to be ascertained along very specific beams.

However, it is also possible within the scope of the invention to specify the height profile in the form of a point cloud. The point cloud comprises a list of points, in each case specifying the respective coordinates thereof in three dimensions in relation to a reference point in space.

Other representations of the relative position of the surface of the person 1 or of the surface of an object situated on the, or next to the, person 1 in relation to the detector unit or in relation to the space are also possible if individual points on this surface are determinable.

The region 25 recorded by the detector unit 20 or covered by the positions is restricted to the body of the person 1 in order to detect as few irrelevant movements of the person 1 or movement artifacts as possible.

The distance measurement values d₁, . . . , dₙ of the height profile H are denoted by H(x,y,t) below, with the first two coordinates denoting the spatial position and the last coordinate t denoting the recording time t₁, . . . , tₚ. It is assumed that xε[0, . . . , X] and yε[0, . . . , Y], i.e. the spatial resolution of the data stream is X×Y. The third coordinate t represents time and denotes the recording time t₁, . . . , tₚ, which is specified in multiples of the temporal sampling density or sampling rate, e.g. 1/30 s for 30 frames/second.

Overall, a three-dimensional data structure is created within the scope of the recording, a two-dimensional matrix data structure being respectively available in the data structure for each of the recording times t₁, . . . , tₚ, the entries of said matrix data structure respectively corresponding to the distance of the person 1 from the reference plane 21 in a region defined by the position of the entries in the matrix data structure. Each matrix data structure created thus has the same size. All matrix data structures respectively contain memory positions for the individual distance measurement values d₁, . . . , dₙ or for values derived therefrom.

The spatial resolution of the matrix data structure may be reduced in an optional step by virtue of a plurality of entries in the matrix data structure being combined to form one entry of a reduced matrix data structure. In the simplest case, this may mean that only individual distance measurement values d are used for forming the reduced matrix data structure and the remaining distance measurement values are discarded. A reduced matrix data structure Hᵣ(x,y,t)=H(ax,by,t) is obtained for integer parameters a and b, the memory requirements of said data structure being (a×b)-times smaller than the memory requirements of the matrix data structure.

In order to obtain a better and more robust result, the matrix data structure may be smoothed or filtered prior to the reduction in resolution. To this end, the two-dimensional Fourier transform of H is calculated in respect of the first two coordinates, this is multiplied by a filter transfer function, this signal is periodized using the parameters X/a and Y/b and the inverse Fourier transform is then calculated in order to obtain the reduced matrix data structure. The above-described sub-sampling is merely a special case thereof with a constant filter transfer function. An average over a number of within rectangular regions of the matrix data structure of size a×b may also be formed with the aid of filtering with subsequent sub-sampling.

In a further step, the recording interval in which height profiles H were created is preferably subdivided into adjoining and non-overlapping time portions of 5 to 10 seconds. Segments S₁, . . . , Sₘ containing the individual ascertained matrix data structures, which were recorded within a time portion, are created. Alternatively, it is also possible for the individual time portions to overlap or for height profiles of individual time ranges not to be contained in any of the segments S₁, . . . , Sₙ.

A data structure Hᵢ(x,y,t) emerges for each of the segments Sᵢ, where xε[0, . . . , X], yε[0, . . . , Y] and tεSᵢ. Moreover, the times at which a segment starts are denoted by T_{3D,i}. What is described below is how respiratory activity may be determined for each individual one of these time blocks and how, optionally, the depth of respiration may also be measured.

An observation region 22 is selected for each segment Sᵢ, said observation region denoting the region or regions of the matrix data structure in which a respiratory movement is expected. This may be preset manually and be selected to be constant over the individual segments Sᵢ.

However, it is also possible to adapt the observation region 22 to movements of the person. However, since the individual segments Sᵢ only contain matrix data structures from time ranges of approximately 10 seconds, an adaptation within a segment Sᵢ is not required in most cases. The observation region 22 is usually considered to be constant within a segment Sᵢ.

Adapting the observation region 22 for the individual segments Sᵢ is advantageous in that a simple detection of the respiration of the person 1 is also possible if they move in their sleep. The observation region 22 is advantageously determined automatically. Three different techniques are described below for automatically adapting the observation region 22 for each segment Sᵢ so as be able to follow possible movements of the person 1.

A temporal signal sᵢ is created after the observation region 22 is set, with a signal value sᵢ(t) respectively being ascertained for each matrix data structure. In the present case, the signal value is ascertained by virtue of the mean value of the distance values H(x,y,t), the positions of which lie within the observation region 22, being ascertained for the respective time t. A signal sᵢ(t) which is only still dependent on the time is determined in this way.

This signal s(t) may possibly contain noise. In order to obtain the respiration and, optionally, a measure for the depth of respiration as well from this noisy signal s(t), said signal s(t) may be subjected to noise filtering. By way of example, advantageous noise suppression may be attained by virtue of signal components with a frequency of more than 1 Hz being suppressed. Alternatively, or additionally, provision may also be made for suppression of direct components or of frequencies up to 0.1 Hz. What this filtering may achieve is that only frequencies which are relevant to determining the respiration remain. The signal sᵢ(t) obtained thus has good correspondence with the actually carried out respiratory movements.

The depth of respiration T may be derived particularly advantageously from this signal s(t). There are many options to this end, two of which are illustrated in more detail. FIG. 2a shows a first signal curve of the signal s(t), with a minimum sₘᵢₙ and a maximum sₘₐₓ being searched for within the signal curve. Subsequently, the difference Δ between the minimum sₘᵢₙ and a maximum sₘₐₓ is formed and this difference Δ is used as a measure for the depth of respiration T.

As may be seen from FIG. 2b, the respiration of the person 1 is significantly weaker than in the signal depicted in FIG. 2a. It is for this reason that the difference Δ between the minimum sₘᵢₙ and a maximum sₘₐₓ is also smaller than in the signal depicted in FIG. 2a.

To the extent that a plurality of local maxima and minima are found within a signal, it is also possible to use the greatest difference between respectively one minimum and the respective next local maximum as depth of respiration T.

Alternatively, it is also possible to subject the signal to a spectral transformation, in particular a Fourier transform, cosine transform or wavelet transform. The spectral component with the highest signal energy is searched for within a predetermined frequency band, in particular from 0.1 Hz to 1 Hz. The signal energy of this spectral component is used for characterizing the depth of respiration T in this segment.

FIG. 3 depicts a sequence of signals sᵢ(t) of temporally successive segments Sᵢ, wherein an interruption of respiration of the person 1 is present during segments S₃ and S₄. Further, over the same time axis, FIG. 3 also respectively illustrates the depth of respiration Aᵢ for the respective segment Sᵢ and an interpolation curve T(t) which specifies the depth of respiration T for all times within the illustrated segments S₁, . . . , S₇. It is clearly visible that the values A₁, . . . , A₇ for the depth of respiration during the interruption of respiration in segments S₃ and S₄ are significantly reduced in relation to the other segments.

Individual possible procedures for automatically identifying the observation region 22 within a segment Sᵢ are shown below.

In a first procedure (FIG. 4), individual possible observation regions R_{1,1}, . . . are predetermined. Subsequently, in each case the depth of respiration T is ascertained in all possible observation regions R_{1,1}, . . . in accordance with the methods specified above. FIG. 5 shows individual signals s_{3,4}, s_{2,3} and s_{3,3}, which were created on the basis of the possible observation regions R_{3,4}, R_{2,3} and R_{3,3}. Since the possible observation region R_{3,3} precisely images the thoracic region of the person 1, the signal amplitudes in this possible observation region R_{3,3} are also larger than in the other two observation windows R_{3,4}, R_{2,3}, which respectively image the head and the arms of the person 1. It is for this reason that the possible observation region R_{3,3} is selected as observation region 22 for the respective segment Sᵢ.

The position and size of the observation region R_{3,3} may still be improved by virtue of attempts being made to displace the corners of the rectangle setting the observation region and recalculate the depth of respiration T on the basis of this modified observation region. By varying the corners of the observation region, the latter may be adaptively improved until it is no longer possible to achieve an increase in the ascertained depth of respiration by displacing the corners.

A further procedure for determining the observation region 22, depicted in FIG. 6, may be undertaken by means of object recognition. Initially, regions 31, 32 corresponding to a human head and upper body or torso are searched for in one or more of the matrix data structures of the segment Sᵢ by means of object recognition. The region 32 of the matrix data structure mapping the torso, or a portion of this region, may be selected as observation region 22.

The portion 33 of this region 32 close to the head 31 may be selected as observation region 22 for the purposes of detecting costal respiration. The portion 34 of this region 32 away from the head 31 may be selected as observation region 22 for the purposes of detecting diaphragmatic respiration.

A number of different image processing methods may be used for the object recognition. The goal of known object recognition methods lies in identifying the contours of the human body automatically or semi-automatically, i.e. partly assisted by humans, in a 3D image or height profile. Such a procedure is described in the following publications:

Gabriele Fanelli, Juergen Gall, and Luc Van Gool, Real time head pose estimation with random regression forests, in Computer Vision and Pattern Recognition (CVPR), pages 617-624, June 2011.

Jamie Shotton, Ross Girshick, Andrew Fitzgibbon, Toby Sharp, Mat Cook, Mark Finocchio, Richard Moore, Pushmeet Kohli, Antonio Criminisi, Alex Kipman, et al., Efficient human pose estimation from single depth images, Pattern Analysis and Machine Intelligence, IEEE Transactions on, 35(12):2821-2840, 2013.

Statistical learning methods are used in the detection of body structures by means of object recognition. While the first publication targets determination of the pose of the head, the second publication targets the localization of the body and, specifically, the joints. The position of the head and the torso in the height profile H may also be determined using both methods.

A further option consists of initially setting the height profile H of the head manually and then determining where the head is situated at said time in each segment Sᵢ with the aid of correlation. The torso may also be found in the same way in each segment Sᵢ or in each matrix data structure within a segment Sᵢ. If the positions of the torso and of the head are known, it is easy to subdivide the torso into the chest and abdominal region. As a result, it is possible to detect respiration separately in both the chest and in the abdominal area. The same segmentation of the body of the person may be realized with the aid of many different algorithms.

If it is possible to identify an observation region 22 in one of the segments Sᵢ, the observation region 22 may be identified in most cases with little outlay in the segment Sᵢ₊₁ which immediately follows this segment Sᵢ. In both procedures illustrated above, knowledge of the approximate position of the image of the abdominal or chest region simplifies finding the abdominal or chest region in the respectively next segment Sᵢ₊₁.

FIGS. 7 and 8 illustrate a third option for finding the abdominal or chest region of the person as observation region 22. In a first step, the variance of the values over the respective segment Sᵢ is determined separately for all entries of the matrix data structure. Subsequently, the region or contiguous regions of entries within the matrix data structure whose respective variances lie above a lower threshold or within a predetermined interval are selected as observation region 22. FIG. 7 shows a section through a height profile of a person along the sectional line VII-VII from FIG. 2. The dashed line shows a temporal mean value of the individual distance values at the respective position. The arrows show the variance at the respective position. It is possible to see that greater variances occur in the region of the chest and in the region of the abdomen of the person than in the remainder of the body. These regions are selected as observation regions 22a, 22b.

In order to be able to avoid erroneous detections by individual artifacts, only contiguous regions 22a, 22b of the matrix data structure whose size exceeds a predetermined number of entries are selected as observation region.

This variant of ascertaining the observation region 22 also permits the detection of changes in position of the person and the detection of measurement errors. If the observation region 22 is ascertained separately in two or more successive segments Sᵢ, . . . , a height profile may be discarded and an error message may be output if the size and the centroid of an observation region 22a, 22b shifts or changes by a predetermined threshold in relation to the corresponding observation region 22a, 22b in the respective preceding segment Sᵢ.

Naturally, it is also possible to combine the aforementioned procedures for determining the observation region 22. By way of example, the torso may be determined with the aid of image processing methods for image recognition and then only those entries within this region for which a significant movement was detected with the aid of the variance may be selected. Alternatively, it is also possible to select a plurality of different possible observation regions 22 and then, for respiration detection purposes, use the observation region 22 with which the greatest depth of respiration was ascertained.

In the illustrated exemplary embodiments, use is always made of a height profile which is stored in the matrix data structure. However, the invention also allows alternative embodiments, in which the height profiles in each case only set a number of points situated in space. It is not necessary for the respective points to be set explicitly by coordinates for as long as the respective point is uniquely settable in space on account of the specifications in the height profile H. This is given implicitly in the present exemplary embodiments by the specific position at which the distance of the person 1 is determined.

Alternatively, it is also possible to specify the height profile by way of a point cloud, i.e. substantially a list of points in space. In such a case, the observation region 24 may be set as a three-dimensional region which is situated in the chest or abdominal region of the person 1. For the purposes of forming the respective mean value, use is only made of points situated within the respective observation region 22. In order to cover all instances of the chest or abdominal region of the person 1 rising and falling, a region which also comprises a volume lying above the chest or abdominal region, up to approximately 10 cm above the chest or abdominal region, may be selected as observation region 22.

A preferred embodiment of the invention also provides the option of ascertaining the depth of respiration and respiration of the person 1 by acoustic recordings in parallel with the height profiles. FIG. 1 depicts an optional microphone 40, by means of which an audio signal sₐ is recorded. The audio signal sₐ created by the microphone 40 is digitized and stored with a predetermined sampling rate and kept available. The microphone is connected to the processing unit 50.

The audio signal sₐ is initially subdivided into audio segments Sₐ with a predetermined length of approximately 200 ms. In general, these audio segments Sₐ may also overlap; however, in the present case, adjoining, non-overlapping audio segments Sₐ are selected.

Optionally, the audio segments Sₐ may be multiplied by a window function in order to avoid edge effects in the Fourier methods described further below. Moreover, a time T_{audio,i} is also linked to each audio segment Sₐ, said time specifying the time at which the segment fᵢ starts.

Each audio segment S_{Ai} is fed to a classification which is used to identify whether no noise, a background noise H, a respiratory noise or a snoring noise A/S may be heard in the respective audio segment S_{Ai}. A classification result having a value N, H, A/S (FIG. 9) relating to whether no noise, a background noise, a respiratory noise or a snoring noise can be heard is respectively created for each audio segment S_{Ai}. The classification results are combined to form a discrete temporal classification signal A(t).

The following describes how each of these individual audio segments S_{Ai} may be examined for the presence of snoring noises using statistical learning methods.

In a first step, a feature vector mᵢ may be extracted from the audio segments S_{Ai} for the purposes of detecting snoring noises in the individual audio segments S_{Ai}. The feature vector mᵢ for the i-th segment audio segment S_{Ai} is calculated directly from the sampling values of the respective audio segment S_{Ai}. The i-th feature vector mᵢ may have different dimensions depending on which methods are used for calculating the features. Some different techniques which may be used to generate the features are listed below.

A spectral analysis of the respective audio segment S_{Ai} may be carried out with the aid of the Fourier transform of an audio segment S_{Ai}. The energies in specific frequency bands, i.e. the sum of the squares of the magnitude of the Fourier coefficients of certain pre-specified bands, are used as features. As a result, a vector with a length which is set by the number of bands is obtained for each audio segment S_{Ai}. If another discrete cosine transform is applied to this vector, a further possible set of coefficients of the feature vector mᵢ is obtained.

A further possible feature of a feature vector mᵢ is the energy in an audio segment S_{Ai}. Moreover, it is also possible to use the number of zero crossings, i.e. the number of sign-changes in an audio segment S_{Ai}, as a possible feature of a feature vector mᵢ.

After the composition of a feature vectors mᵢ has been set, a statistical learning method is selected. To this end, a large number of possible methods are available in the prior art, and so the various options are not discussed in any more detail here. These methods allow an audio segment S_{Ai} to be identified as a snoring noise on the basis of a feature vector mᵢ. To this end, the feature space is subdivided into a set of points which are assigned to the snoring and into the rest which are classified as not snoring. In order to be able to undertake this classification, these algorithms are initially trained on the basis of known snoring noises; i.e., audio segments with snoring noises are selected manually, the feature vectors mᵢ thereof are calculated and the classification algorithm is then trained therewith. Implementations of all employed statistical learning methods are freely available and already implemented.

By means of the aforementioned classification methods, it is possible to determine which audio segments S_{Ai} contain snoring noises. However, an average snoring signal often also has quiet time intervals between the loud phases since snoring is often only carried out during inspiration, as shown in FIG. 9. In order to be able to correctly assign the entire snoring phase and also the quiet segments between the loud episodes to snoring, a plurality of segments are considered beforehand and afterward at the i-th position. If more segments with snoring noises than a certain threshold are contained in this set, snoring is detected at the time T_{audio,i}.

The goal of classifying recorded noises in order to be able to distinguish respiratory noises such as snoring and respiration from other possibly occurring background noises may be achieved using methods known from the prior art. In particular, such methods are known from the following publications:

Hisham Alshaer, Aditya Pandya, T Douglas Bradley, and Frank Rudzicz. Subject independent identification of breath sounds components using multiple classifiers. In Acoustics, Speech and Signal Processing (ICASSP), 2014 IEEE International Conference on, pages 3577-3581, IEEE, 2014.

Different statistical learning methods are used in order to be able to classify the recorded noises into various categories. In the first paper, the microphone is fixed in a mask on the face of the patient; as a result, there are hardly background noises or these are negligible. In this publication, the recorded noises are classified as inspiration, expiration, snoring and panting. To this end, a multiplicity of different features are generated, by means of which the selected learning method operates.

M Cavusoglu, M Kamasak, O Erogul, T Ciloglu, Y Serinagaoglu, and T Akcam, An efficient method for snore/nonsnore classification of sleep sounds, Physiological measurement, 28(8):841, 2007.

Statistical learning methods are also used in this publication in order to be able to categorize the recorded noises into various categories. The microphone is at a distance of approximately 15 cm from the head of the subject and the recorded noises are subdivided into “snore” or “nonsnore”.

FIG. 9 depicts snoring noises Xₛ during expiration, with background noises X_{H} also being recorded between these snoring noises Xₛ. On account of the illustrated classification, one classification value Kₐ may be assigned in each case to each one of the audio segments S_{Ai}, said classification value being depicted in the uppermost region of FIG. 9.

Depending on the quality of the audio signal and the level of the background noises, the snoring noises may optionally be classified more precisely into subclasses, such as e.g. pathological and non-pathological. Respiration may also be classified as a separate noise under very expedient conditions, and the strength thereof may be ascertained.

FIG. 9 depicts the result of an improved embodiment of the invention in more detail, in which both the results of the evaluation of the height profiles and the results of the acoustic snoring analysis are included in the evaluation of the respiratory activity. Typically, the segments Sᵢ and the audio segments S_{Ai} are not correlated with one another in time.

For this reason, audio segments S_{Ai} and segments Sᵢ which were recorded at the same time are initially assigned to one another on account of their respective recording time. Typically, a plurality of audio segments S_{Ai} are available for each segment Sᵢ, said audio segments being recorded during the time interval of the segment.

Segments Sᵢ with a low depth of respiration T are searched for in the present exemplary embodiment of the invention. What may be gathered from the data illustrated in FIG. 9 is that a depth of respiration T in the segment S₃ is substantially lower than in the two preceding segments S₁, S₂. Subsequently, missing respiratory noises are searched for in all audio segments S_{Ai} assigned to this segment S₃. Here, it is possible to determine that no snoring or respiratory noises, but merely background noises X_{H}, are present in the audio segments S_{Ai} which were recorded at the same time as the segment S₃. Since, firstly, a low depth of respiration was identified and, secondly, it has not been possible to determine any snoring or respiratory noises either, the assumption may be made the relevant person is not breathing.

Statements about the respiration now may be made at any time from the data which were extracted from the audio signals and the height profiles. To this end, the following procedure is carried out: two indices I and J are searched for such that the respective start times T_{audio,I} of the audio segments S_{Ai} and the start times T_{3D,J} of the segments S are respectively closest at the time t from all possible times which were selected for the audio segmentation and for the segmentation of the height profiles. The options emerging for this time are summarized in the following table:

| | Snoring at T_{audio, I} | No snoring at T_{audio, I} |
|---|---|---|
| Deep depth of | Respiration | Respiration |
| respiration at T_{3D, J} | | |
| No or low depth of | Respiration | Apnea |
| respiration at T_{3D, J} | | |

Below, a further embodiment of the invention is illustrated, in which an improved linking of the ascertained depth of respiration T and the acoustic classification of the respiratory noises and hence an improved detection of interruptions of respiration facilitated. It is possible to ascertain a depth of respiration signal T(t) by interpolating the ascertained depth of respiration over time.

This classification is based on the depth of respiration function T(t), which denotes the time curve of the depth of respiration, and the classification signal A(t), which specifies whether, and optionally which, respiratory noises were present at any one time.

In the present exemplary embodiment, the classification signal A(t) is modified and set as a vector-valued function which assigns a vector to each time, the components of said vector respectively specifying the intensity of the respectively identified signal. Specifically, the classification signal A(t) could have components, of which one represents the strength of the snoring noise or of a snoring noise of a specific type, while another one of which represents the strength of the background noises present at the respective time.

Then, it is possible to combine a plurality of audio segments to form a further audio segment with a length of, for example, 5 to 10 seconds and ascertain the respective signal strengths in this further audio segment by forming an average. As a result of this step, it is possible to classify relatively long periods, in which a person snores during inspiration but expires quietly, as snoring overall. An averaged classification signal B(t) is created by this averaging. It is particularly advantageous if the averaged classification signal B(t) and the depth of respiration signal A(t) are set on temporally corresponding segments or further audio segments.

In a subsequent step, times where strong changes occur in both signals T(t), B(t) are searched for in both the depth of respiration signal T(t) and in the further classification signal B(t). By way of example, these are times at which respiratory noises disappear or return, or respiratory movements disappear or return.

The times identified thus are considered ever more relevant with increasing strength of the change in the signal T(t), B(t) at the respective time. A relevance value REL is created in this respect, said relevance value specifying the relevance of the individual times for the start or the end of a phase with little respiration.

Those points for which the magnitude of the relevance measure REL lies over the magnitude of a predetermined threshold are selected as start points R_{S} or end points R_{E} of apnea. A start time R_{S} may be assumed if the relevance value REL is negative; an end time R_{E} may be assumed if the relevance value REL is positive. The period of time AP between the start time R_{S} and the end time R_{E} is considered to be a period of time with little or lacking respiratory activity.

In particular, the threshold may be formed in a looping or sliding manner by virtue of a mean value of the reference measure being formed over a time range, in particular before and/or after the comparison time with the reference measure, and the threshold being set in the range between 120% and 200% of this mean value.

Overall, it is possible within the scope of the invention to record the entire sleep of a person over several hours and subdivide the height profiles, optionally the audio signals as well, into individual segments, and optionally into audio segments as well. Naturally, it is also possible to consider only individual particularly relevant phases of sleep of the person and only assign some of the recorded height profiles or audio sampling values to individual segments and optionally audio segments.

Further, it is possible to record the number and duration of the ascertained phases of low depth of respiration or of lacking respiratory activity over the entire sleep and to subject these to statistical analyses.

Below, further preferred embodiment variants of the invention are illustrated in more detail. As a matter of principle, time ranges which, in regions relevant to the respiration, are free from movements which cannot be traced back to the respiration and which originate from other movements of the person during the sleep are initially ascertained in these embodiment variants. Subsequently, regions of the height profile in which the respiration of the person may be ascertained best are identified. These regions are subsequently used for creating a depth of respiration signal.

During the sleep, there occasionally are upper body movements, such as e.g. rolling movements, of the sleeping person. Such movements may easily be identified as these movements are far more pronounced than respiratory movements, which only have a small movement amplitude and moreover are far more regular. Time ranges between the individual movements are identified in order to avoid that such movements with a relatively large amplitude result in discontinuous changes of the ascertained signals in the ascertained height profiles; depth of respiration measurements are ascertained separately for these time ranges. Each time range obtained in this manner is assigned to a separate depth of respiration curve in each case. In the process, it is possible to resort to the aforementioned procedure according to the invention.

The ascertained time ranges, which are free from relatively large movements of the person are subdivided into individual segments which each have a number of height profiles H recorded at successive recording times t1, . . . , tp, in particular within a time range of 3 to 20 seconds.

The observation region 22 may advantageously be selected by virtue of time signals for the segments of the time range being initially created separately for all points of the height profile H, in particular for all entries of the matrix data structure or the point cloud, said time signals specifying the time curve of the height profile H in the respective point within the respective segment.

A value characterizing the respiration or the strength of respiration is derived from each of these time signals and assigned to the respective point of the height profile. The depth of respiration value may be derived in different ways. In addition to using the variance, it is also possible to advantageously use specific components of the ascertained signal spectrum for the purposes of ascertaining the depth of respiration.

To this end, the signal energy within two predetermined frequency ranges is respectively ascertained for each entry of the height profile. The first frequency range, by means of which the presence and the strength of respiration may be accurately estimated and which is characteristic for the strength of the respiration, lies approximately between 10/min and 40/min. Advantageously, the lower limit of this first frequency range may generally advantageously lie between 5/min and 15/min; the upper limit of the first frequency range advantageously lies between 25/min and 60/min.

However, these specified limits are only exemplary and depend very strongly on the age of the respective patient. By way of example, infants breathe three times as fast as adults. In principle, these limits or thresholds emerge from the lowermost assumed value for the respiratory frequency, which lies at approximately 10/min for adults. The upper limit may be set to 3 to 4 times the lower limit value, i.e. to 30/min to 40/min, in particular also in order to take into account harmonics of the respiratory movements into account.

A signal noise is likewise ascertained; it may be able to be determined by signal components within a second frequency range which, in particular, lies between 360/min and 900/min. In general, the lower limit of this second frequency range may advantageously lie between 180/min and 500/min; the upper limit of the second frequency range advantageously lies between 600/min and 1200/min. Setting this frequency range also depends strongly on the examined patient. In principle, use is made of a frequency band which lies above the first frequency range and which has no influence on the respiration of the patient.

Subsequently, the quotient of the signal energy in the first frequency range and the energy of the measurement noise in the second frequency range is ascertained by the processing unit 50; it is used as respiratory strength value for characterizing the presence of respiration.

Subsequently, a region or a plurality of regions 22a, 22b with respectively contiguous points of the height profile H, the respective depth of respiration value of which lies above a lower threshold or within a predetermined interval, is/are selected as observation region 22 for each one of the segments. This may preferably be carried out by virtue of contiguous regions 22a, 22b of points whose size exceeds a predetermined number of points being selected as observation region.

A region may be considered contiguous if each point of the region is reachable via respectively adjacent pixels proceeding from another point within this region. Preferably, points of the height profile H may be considered to be adjacent
if they are set by adjacent entries of the matrix data structure or
if they, or the projection thereof onto a horizontal plane in space, have a distance from one another which is less than a threshold.

The relationship may be set in different ways; in particular, it is possible, for the purposes of setting the neighborhood within the height profile, to set those points as neighbors which differ from one another by at most a value of 1 in at most one index. This is also referred to as a 4-neighborhood.

The respective threshold may be set dynamically, facilitating an adaptation to different sleep postures. The threshold is adapted if pixel number of the largest contiguous region does not lie within a predetermined threshold range. If the pixel number of the largest range is greater than the maximum value of the threshold range, the threshold is doubled; if the pixel number is less than the minimum value of the threshold range, the threshold is halved.

The region 22 with the largest number of pixels is selected in each case for each temporal segment. Alternatively, it is also possible to select a number N of the largest regions 22. The centroid of the region 22 is stored in each case for each temporal segment. The median mx is subsequently formed in each case within the time range over all available x-coordinates of centroids. Likewise, the median my is formed in each case within the time range over all available y-coordinates of centroids. Subsequently, the region whose centroid has the smallest distance from the point whose y-coordinate corresponds to the median my of the y-coordinates and whose x-coordinate corresponds to the median mx of the x-coordinates is ascertained from all regions 22 ascertained for the time range.

This region is subsequently used for ascertaining the depth of respiration signal for the entire time range. The depth of respiration signal is ascertained by virtue of the mean value or the sum of the values of the height profile H being ascertained within this region, respectively separately for each time. Where necessary, the depth of respiration signal ascertained thus may still be subjected to low-pass filtering, with use being made of a filter frequency between 25/min and 60/min. When setting the filter frequency or limit frequency of the low-pass filter, the precise values also depend strongly on the age of the patient. In addition to low-pass filtering, use may also be made of de-trending or high-pass filtering.

1-43. (canceled)

44. A method for determining a time curve of a depth of respiration of a person, which comprises the steps of:
using a detector unit directed to the person on an ongoing basis for in each case creating a height profile of the person at successive recording times;
setting in space a number of at least two points in the height profile, the points lying on a surface of the person or on a surface of an object situated on, or next to, the person;
storing the height profile for each of the successive recording times in a data structure;
combining a number of height profiles recorded at the successive recording times to form a segment;
selecting a region which specifies an abdominal region or a chest region of the person depending on a respective reference point or reference region as an observation region;
ascertaining a mean value of distances of the points of the height profile situated within the observation region from the respective reference point or the reference object, separately in each case, for each said height profile within the segment;
ascertaining a signal for the segment, the mean value ascertained for the height profile at a respective recording time of the height profile being assigned to the signal; and
ascertaining at least one value characterizing the time curve of the depth of respiration on a basis of the signal.

45. The method according to claim 44, wherein the height profile describes a point cloud with a number of at least two points in space, the points lying on the surface of the person or on the surface of an object situated on, or next to, the person.

46. The method according to claim 44, which further comprises extracting at least one maximum and at least one minimum from the signal for purposes of characterizing the depth of respiration in the segment and at least one difference between the maximum and the minimum is used as the value characterizing the depth of respiration for a time range assigned to the segment.

47. The method according to claim 44, which further comprises subjecting the signal to a spectral transformation and a spectral component with a highest signal energy is searched for within a predetermined frequency band, and the signal energy of the spectral component is used to characterize the depth of respiration in the segment.

48. The method according to claim 44, which further comprises subjecting the signal assigned to the segment to noise filtering after a creation thereof, prior to determining the value characterizing the depth of respiration, wherein, signal components with a frequency of more than 0.5 Hz are suppressed and/or direct components are suppressed.

49. The method according to claim 44, which further comprises:
ascertaining the depth of respiration separately in each case for a number of overlapping or non-overlapping segments; and/or
ascertaining the observation region for each of the segments separately on a basis of the observation region ascertained for a respective preceding segment.

50. The method according to claim 44, wherein:
the height profile is characterized by a two-dimensional matrix data structure containing a number of rows and columns;
a number of positions disposed in a grid-shaped manner in lines and columns are predetermined, with distance values of the height profile being determined at the positions in each case;
the distance values in particular being set along predetermined rays, which emanate from the detector unit, or as normal distances from a respective point of intersection of a beam with the surface to the reference plane with an aid of a measured distance and a respectively employed measurement angle;
measurement angles of the rays are selected in such a way in a process that a grid-shaped arrangement emerges upon incidence of the rays on a plane lying parallel to the reference plane, the matrix data structure having a grid with a same size and structure; and
the matrix data structure is created by virtue of the distance values recorded at respective positions being stored and being kept available at memory positions, corresponding to the positions in the grid, in the matrix data structure.

51. The method according to claim 50, which further comprises replacing the two-dimensional matrix data structure, after a creation thereof, in its dimensions by a reduced matrix data structure, wherein a mean distance value is ascertained in each case for rectangular and non-overlapping image regions, which cover an entire matrix data structure, having the same size in the two-dimensional matrix data structure in each case and the mean distance value is assigned to an image point, corresponding in terms of a position thereof, in the reduced matrix data structure.

52. The method according to claim 50, wherein:
the two-dimensional matrix data structure, after a creation thereof, is created in its dimensions by a reduced matrix data structure, a spatial resolution of which is reduced by virtue of a plurality of entries in the two-dimensional matrix data structure being combined to form one entry in the reduced data matrix structure;
only individual distance measurement values are used for forming the reduced matrix data structure and remaining distance measurement values are discarded; and
parameters are determined as integer values and the reduced matrix data structure is determined according to Hᵣ(x,y,t)=H(ax,by,t).

53. The method according to claim 50, wherein the two-dimensional matrix data structure, after a creation thereof, is replaced in its dimensions by a reduced matrix data structure, wherein a distance value being a mean distance value, is ascertained for regions of the two-dimensional matrix data structure and the distance value is assigned to an image point, corresponding in terms of position thereof, in the reduced data matrix structure.

54. The method according to claim 44, wherein the observation region is placed by virtue of:
a number of possible observation regions being predetermined in advance in the height profile;
a respective depth of respiration being ascertained for the respective segment on a basis of each one of possible observation regions; and
a predetermined possible observation region with a largest ascertained depth of respiration being used as the observation region.

55. The method according to claim 50, wherein the observation region is placed by virtue of:
regions being searched for in the height profile by means of object recognition, or in that the regions are selected in advance, the regions corresponding to a human head and torso; and
the region of the height profile imaging the torso or a portion of the region being selected as the observation region, wherein a portion of the region close to the head is selected as the observation region for purposes of detecting the curve or the depth of respiration of a costal respiration, and/or a portion of a region away from the head is selected as the observation region for detecting the curve or the depth of respiration of a diaphragmatic respiration.

56. The method according to claim 55, wherein for each of the segments to be examined or for individual height profiles of the segment, the observation region is adaptively ascertained anew by means of object recognition applied to the height profiles present in the segment, proceeding from a position of a selected observation region in a respectively preceding segment.

57. The method according to claim 50, wherein the observation region is placed by virtue of:
a time signal being separately created for a time interval for all the points of the height profile, the time signal specifying a time curve of the height profile at a respective point, and a respiration strength value characterizing the depth of respiration being respectively derived from the time signal assigned to the respective point of the height profile;
at least one region with respectively contiguous points of the height profile, a respective respiratory strength value of which lies above a lower threshold or within a predetermined interval, being selected as the observation region, by virtue of contiguous regions of points being selected as observation region, a size of which exceeds a predetermined number of points, wherein:
a region is considered to be contiguous if each point of the region is reachable via respectively adjacent pixels proceeding from another point within the region; and
the points of the height profile are considered to be adjacent if they are set by adjacent entries of the two-dimensional matrix data structure or if they, or the projection thereof onto a horizontal plane in space, have a distance from one another which is less than a threshold.

58. The method according to claim 57, which further comprises using a variance of the time signal as the respiratory strength value.

59. The method according to claim 57, wherein for creating the respiratory strength value:
ascertaining a level of a signal energy in a first frequency range between 10/min and 40/min;
ascertaining a level of the signal energy in a second frequency range between 360/min and 900/min; and
ascertaining a quotient of the signal energy in the first frequency range and the signal energy in the second frequency range, the quotient being used as the respiratory strength value.

60. The method according to claim 44, which further comprises:
ascertaining the observation region separately in at least two successive segments and the height profile is discarded and an error message is output if the observation region is displaced by a predetermined threshold in relation to a respective preceding segment.

61. The method according to claim 44, which further comprises:
ascertaining a sound emitted by the person simultaneously and in parallel with a distance of the person from the detector unit being recorded, the sound being kept available in a form of an audio signal with a number of audio sampling values;
subdividing the audio signal into a number of audio segments and carrying out an examination for each of the audio segments as to whether the audio segments contain human respiratory noises or other noises, and a classification result is respectively kept available for each of the audio segments;
assigning the audio segments and the segments recorded at a same time to one another;
searching for the audio segments or the segments with a low depth of respiration or a lack of respiratory noises and the segments or the audio segments assigned to such an audio segment or the segment are likewise examined for a presence of the low depth of respiration or the lack of respiratory noises; and
determining a lack of respiration of the person should the low depth of respiration and the lack of respiratory noises be detected in each case in segments and the audio segments that are assigned to one another.

62. The method according to claim 61, wherein:
a classification signal is created, the classification signal in each case containing at least one classification result for each of the audio segments, the classification result specifying a type and strength of a respective noise in a time range of a respective audio segment;
in that in each case a number of successive audio segments are combined to form further audio segments which contain same time ranges as the segments;
averaging is undertaken over the classification results contained within a further audio segment and a respective mean value is assigned to the further audio segment;
a further classification signal is created by interpolating mean values of the further audio segments;
times at which strong changes occur in both signals are searched for in a further classification signal and in a depth of respiration signal;
the times identified thus are classified as ever more relevant with increasing strength of a change in the respective signal at the respective time;
a relevance value in this respect is assigned to these times in each case; and
the times for which a magnitude of a relevance value lies above a magnitude of a threshold are detected as start points or end points of apnea, wherein the threshold is formed by virtue of a mean value of the reference measure being formed over a time range, before and/or after a comparison time with a reference measure, and the threshold being set in a range between 120% and 200% of the mean value.

63. The method according to claim 44, which further comprises:
ascertaining a time range or a plurality of time ranges, in which a change in the height profile does not exceed a predetermined threshold;
using only the successive recording times from a same time range for determining the curve of the depth of respiration; and
carrying out where necessary, a determination of the curve of the depth of respiration separately for a plurality of ascertained time ranges if the plurality of time ranges are present.

64. A method for determining a time curve of a depth of respiration of a person, which comprises the steps of:
using a detector unit directed to the person on an ongoing basis for in each case creating a height profile of the person at successive recording times;
providing the height profile with a number of at least two distance values for in each case setting one point in space, wherein individual distance values in each case specify a distance of a point of intersection of a beam and a surface of the person or a surface of an object situated on the, or next to the, person from a reference point or a reference plane, the beam being set in advance relative to the detector unit;
creating a data structure in each case for each of the successive recording times, the data structure containing the height profile, wherein all data structures thus created have a same size in each case and each have with memory positions for individual distance values of the height profile;
combining a number of height profiles recorded at successive recording times to form a segment;
selecting an observation region from a number of the memory positions in the data structure, in which the distance values specifying a distance of an abdominal or a chest region of the person depending on the reference point or the reference region are stored;
ascertaining a mean value of the distance values situated within the observation region, separately in each case, for each of the height profiles within a segment;
ascertaining a signal for the segment, the mean value ascertained for the height profile at a respective recording time of the height profile being assigned to said signal; and
ascertaining at least one value characterizing the time curve of the depth of respiration on a basis of the signal.

65-83. (canceled)

84. A non-transitory data medium having computer executable instructions for executing the method according to claim 44.

85. (canceled)

86. An apparatus for determining a time curve of a depth of respiration of a person, the apparatus comprising:
a detector unit alignable onto a space to sleep, said detector unit, on an ongoing basis, in each case creating a height profile of the person for successive recording times, wherein a number of at least two points are set in space in the height profile, said points lying on a surface of the person or on a surface of an object situated on, or next to, the person; and
a processing unit, programmed to:
store and keep available the height profile in a data structure for each of the successive recording times;
combine a number of height profiles recorded at the successive recording times to form a segment;
select as an observation region a region which specifies an abdominal or chest region of the person depending on a respective reference point or reference region;
ascertain a mean value of distances of the points of the height profile situated within the observation region from the reference point or the reference object, separately in each case, for each of the height profiles within the segment;
ascertain a signal for the segment, the mean value ascertained for the height profile at a respective recording time of the height profile being assigned to the signal; and
ascertain a value characterizing the depth of respiration on a basis of the signal or a signal amplitude of the signal.

87. The apparatus according to claim 86, wherein said detector unit creates the height profiles in a form of point clouds with a number of at least two points in space, wherein the points lie on the surface of the person or on the surface of an object situated on, or next to, the person.

88. The apparatus according to claim 86, wherein said processing unit extracts at least one maximum and at least one minimum from the signal for characterizing the depth of respiration in the segment and keeps available and, where required, uses at least one difference between the maximum and the minimum as the value characterizing the depth of respiration for a time range assigned to the segment.

89. The apparatus according to claim 86, wherein said processing unit subjects the signal to a spectral transformation, and searches for a spectral component with a highest signal energy within a predetermined frequency band, and uses the signal energy of the spectral component to characterize the depth of respiration in the segment.

90. The apparatus according to claim 86, wherein said processing unit subjects the signal assigned to the segment to noise filtering after a creation thereof, prior to determining the value characterizing the depth of respiration, wherein, said processing unit further programmed to:
suppress signal components with a frequency of more than 0.5 Hz; and/or
suppresses direct components.

91. The apparatus according to claim 86, wherein said processing unit:
ascertains the depth of respiration separately in each case for a number of overlapping or non-overlapping segments; and/or
ascertains the observation region for each of the segments separately on a basis of the observation region ascertained for a respective preceding segment.

92. The apparatus according to claim 86, wherein said processing unit:
characterizes the height profile by a two-dimensional matrix data structure containing a number of rows and columns, a number of positions disposed in a grid-shaped manner in lines and columns are predetermined, with distance values of the height profile determined at said positions in each case, the distance values are determined as the distance values along predetermined rays, which emanate from said detector unit, or as normal distances from a respective point of intersection of a beam with a surface to the reference plane with an aid of a measured distance and a respectively employed measurement angle, wherein said processing unit selects measurement angles of the predetermined rays in such a way in a process that a grid-shaped arrangement emerges upon incidence of the predetermined rays on a plane lying parallel to the reference plane, the two-dimensional matrix data structure has a grid with a same size and structure, and creates the matrix data structure by virtue of it storing and keeping available the distance values recorded at respective positions at memory positions, corresponding to the positions in the grid, in the two-dimensional matrix data structure.

93. The apparatus according to claim 92, wherein:
said processor unit replaces the two-dimensional matrix data structure, after a creation thereof, in its dimensions by a reduced matrix data structure; and
said processor unit ascertains a mean distance value in each case for rectangular and non-overlapping image regions, which cover an entire matrix data structure, having a same size in the two-dimensional matrix data structure in each case and assigns the mean distance value to an image point, corresponding in terms of the position thereof, in the reduced matrix data structure.

94. The apparatus according to claim 92, wherein said processing unit replaces the two-dimensional matrix data structure, after a creation thereof, in its dimensions by a reduced matrix data structure and reduces its spatial resolution by virtue of combining a plurality of entries in the two-dimensional matrix data structure to form one entry in the reduced data matrix structure, wherein said processing unit only uses individual distance measurement values for forming the reduced matrix data structure and discards remaining distance measurement values, said processing unit determines parameters as integer values and sets the reduced matrix data structure according to Hᵣ(x,y,t)=H(ax,by,t).

95. The apparatus as claimed in claim 92, wherein said processing unit replaces the two-dimensional matrix data structure, after the creation thereof, in its dimensions by a reduced matrix data structure, said processing unit ascertains a distance value for regions of the two-dimensional matrix data structure and assigns the distance value to an image point, corresponding in terms of position thereof, in the reduced data matrix structure.

96. The apparatus according to claim 92, wherein said processing unit sets the observation region by virtue of said processing unit performing the following steps of:
predetermining a number of possible observation regions in advance in the in the height profile;
ascertaining a respective depth of respiration for a respective segment on a basis of each one of possible observation regions; and
selecting and using predetermined possible observation region with a largest ascertained depth of respiration as the observation region.

97. The apparatus according to claim 92, wherein said processing unit sets the observation region by virtue of said processing unit performing the further steps of:
searching for regions in the height profile by means of object recognition, or providing means for selecting regions, the regions corresponding to a human head and torso;
selecting the region of the height profile imaging the torso or a portion of the region as the observation region;
selecting a portion of the region close to the head as the observation region for detecting the curve or the depth of respiration of costal respiration; and/or
selecting a portion of a region away from the head as the observation region for detecting the curve or the depth of respiration of diaphragmatic respiration.

98. The apparatus according to claim 97, wherein said processing unit adaptively ascertains, for each of the segments to be examined or for individual height profiles of the segment, the observation region anew by means of object recognition applied to the height profiles present in the segment, proceeding from a position of a selected observation region in a respectively preceding segment.

99. The apparatus according to claim 92, wherein said processing unit sets the observation region by virtue of said processing unit performing the further steps of:
separately creating a time signal for a time interval for all the points of the height profile, the time signal specifying a time curve of the height profile at a respective point, and respectively deriving a respiration strength value characterizing the depth of respiration from the time signal and assigning the respiration strength value to the respective point of the height profile; and
selecting one region or a plurality of regions with respectively contiguous points of the height profile, the respective respiratory strength value of which lies above a lower threshold or within a predetermined interval, as the observation region, by virtue of said processing unit selecting contiguous regions of points as observation region, a size of which exceeds a predetermined number of points, wherein a region counts as contiguous if each point of the region is reachable via respectively adjacent pixels proceeding from another point within the region, wherein the points of the height profile count as adjacent if they are set by adjacent entries of the two-dimensional matrix data structure or if they, or the projection thereof onto a horizontal plane in space, have a distance from one another which is less than a threshold.

100. The apparatus according to claim 99, wherein said processing unit uses a variance of the time signal as the respiratory strength value.

101. The method according to claim 100, wherein for creating the respiratory strength value, said processing unit ascertains:
a level of signal energy in a first frequency range;
a level of the signal energy in a second frequency range; and
a quotient of the signal energy in the first frequency range and the signal energy in the second frequency range and uses the quotient as the respiratory strength value.

102. The apparatus according to claim 100, wherein said processing unit ascertains the observation region separately in at least two successive segments and discards a height profile and, when necessary, outputs an error message if the observation region is displaced by a predetermined threshold in relation to a respective preceding segment.

103. The apparatus according to claim 86,
further comprising a microphone disposed upstream of said processing unit, said microphone keeping available in a form of an audio signal at an output thereof a sound emitted by the person simultaneously and in parallel with a recording of a distance of the person, and the audio signal is supplied to said processing unit; and
said processing unit further programmed to:
subdivide the audio signal into a number of audio segments and examines for each of the audio segments as to whether human respiratory noises or other noises can be heard therein, and respectively keeps available a classification result for each audio segment;
assign the audio segments and the segments recorded at a same time to one another;
search for the audio segments or the segments with a low depth of respiration or a lack of respiratory noises and likewise examines the segments or the audio segments assigned to the audio segment or the segment for a presence of the low depth of respiration or the lack of respiratory noises; and
determine a lack of respiration of the person should the low depth of respiration and the lack of respiratory noises be detected in each case in the segments and the audio segments that are assigned to one another.

104. The apparatus according to claim 103, wherein said processing unit:
creates a classification signal, the classification signal in each case containing at least one classification result for each of the audio segments, the classification result specifying a type and strength of a respective noise in a time range of a respective audio segment;
in each case combines a number of successive audio segments to form further audio segments which contain a same time ranges as the segments;
undertakes averaging over the classification results contained within a further audio segment and assigns a respective mean value to the further audio segment;
creates a further classification signal by interpolating the mean values of the further audio segments;
searches for times at which changes occur in both signals, namely in the further classification signal and in the depth of respiration signal, and classifies the times identified thus as ever more relevant with increasing strength of a change in the respective signal at the respective time, wherein said processing unit assigns a relevance value in respect to the times in each case;
detects the times for which a magnitude of a relevance value lies above a magnitude of a threshold as start points or end points of apnea;
forms a threshold by virtue of forming a mean value of a reference measure over a time range, and setting the threshold in the range between 120% and 200% of the mean value.

105. The apparatus according to claim 86, wherein said processing unit:
ascertains a time range or a plurality of time ranges, in which a change in the height profile does not exceed a predetermined threshold;
only uses successive recording times from a same time range for determining the curve of the depth of respiration; and
where necessary, separately undertakes a determination of the curve of the depth of respiration for a plurality of ascertained time ranges if the plurality of time ranges are present.

106. An apparatus for determining a time curve of a depth of respiration of a person, the apparatus comprising:
a detector unit alignable onto a space to sleep, said detector unit, on an ongoing basis, in each case creating a height profile of the person for successive recording times, the height profile having a number of at least two distance values for in each case setting one point in space, the distance values in each case specify a distance of a point of intersection of a beam and a surface of the person or a surface of an object situated on the, or next to the, person from a reference point or a reference plane, the beam being set in advance relative to said detector unit; and
a processing unit programmed to:
create a data structure in each case for each of the successive recording times, the data structure containing the height profile, wherein all data structures thus created have a same size in each case and each have with memory positions for individual distance values of the height profile;
combine a number of height profiles recorded at the successive recording times to form a segment;
select a number of the memory positions in the data structure, in which the distance values specifying a distance of an abdominal or chest region of the person depending on a respective reference point or a reference region are stored, as an observation region;
ascertain a mean value of the distance values situated within the observation region, separately in each case, for each of the height profiles within the segment and ascertains a signal for the segment, the mean value ascertained for the height profile at a respective recording time of the height profile being assigned to the signal; and
ascertain a value characterizing the depth of respiration on a basis of the signal or a signal amplitude of the signal.

107-125. (canceled)
